(19) United States
(12) Patent Application Publication
(10) Pub. No.: US 2011/0195911 A1
(43) Pub. Date: Aug. 11, 2011

(54) SYNTHETIC ANALOGS OF THE JUXTAMEMBRANE DOMAIN OF IGF1R AND USES THEREOF

(75) Inventors: Nadya I. Tarasova, Frederick, MD (US); Sergey G. Tarasov, Frederick, MD (US)

(73) Assignee: THE UNITED STATES OF AMERICA,AS REPRESENTED BY THE SECRETARY,DEPARTMENT OF HEALTH & HUMAN SERVICES, Bethesda, MD (US)

(21) Appl. No.: 12/934,911

(22) PCT Filed: Mar. 27, 2009
(86) PCT No.: PCT/US09/38620

§ 371 Date: Nov. 2, 2010

Related U.S. Application Data
(60) Provisional application No. 61/040,203, filed on Mar. 28, 2008.

(51) Int. Cl. A61K 38/16; C07K 7/06; C07K 7/08; C07K 14/00; C12N 15/18; C07K 16/22; A61K 31/7052; A61K 38/10; A61K 38/08; C12N 5/00; A61P 35/00

(52) U.S. Cl. 514/19.3; 530/324; 530/325; 530/326; 530/327; 530/328; 530/329; 536/23.5; 530/387.9; 514/44 R; 514/21.3; 514/21.4; 514/21.5; 514/21.6; 514/21.7; 514/21.8; 435/375

(57) ABSTRACT

A peptide or peptidomimetic comprising the amino acid sequence RXGNGV (SEQ ID NO: 1) or the inverse thereof, or comprising at least six contiguous amino acids of the juxtamembrane domain of IGF1R (SEQ ID NO: 43) or inverse thereof, wherein the peptide or peptidomimetic comprises a total of about 50 or fewer amino acids and inhibits IFG-R1 activity, as well as a method of inhibiting a IGF1R in a cell, a method of treating or preventing IGF1R-mediated disease, and related compounds, compositions, and methods.

### SEQUENCE LISTING

Incorporated by reference in its entirety herein is a nucleotide/amino acid sequence listing submitted concurrently herewith.

### BACKGROUND OF THE INVENTION

Insulin-like growth factor 1 receptor (IGF1R) is a member of the receptor tyrosine kinase (RTK) superfamily. The receptor is an established molecular target for the treatment of many tumor types, and significant efforts have been put into the development of inhibitors of IGF1R-mediated signaling. Inhibition using antibodies to the receptor and using small molecule kinase inhibitors represent two major strategies. Small molecule inhibitors, in turn, can be subdivided into three types: those that bind in the ATP-binding site, substrate-binding competitors, and allosteric inhibitors that target pockets outside of the ATP-binding sites and substrate-binding sites.

Antibodies to IGF1R show promising results in clinical trials; however, the design of selective small molecule IGF1R tyrosine kinase inhibitors is complicated by the fact that the kinase domain of the IGF1R shares 85% homology with the insulin receptor (IR), and the ATP binding cleft is 100% conserved. In addition, IGF1R and IR form functional heterodimers, which further complicate the generation of selective inhibitors. Although it has been suggested that inhibition of both receptors might provide an advantage in anti-cancer therapy because both IGF1R and IR can contribute to tumor growth, selectivity leading to significant toxic effects in animals remains a major problem in the successful development of small molecule kinase inhibitors.

### BRIEF SUMMARY OF THE INVENTION

The invention provides a peptide or peptidomimetic comprising the amino acid sequence RXGNGV (SEQ ID NO: 1) or the inverse thereof, wherein the peptide or peptidomimetic comprises about 50 or fewer amino acids and inhibits IGF1R activity. In another aspect, the invention provides a peptide or peptidomimetic comprising at least six contiguous amino acids of the juxtamembrane domain of IGF1R (e.g., SEQ ID NO: 43), or inverse thereof, wherein the peptide or peptidomimetic comprises a total of about 50 or fewer amino acids and inhibits IFG-R1 activity. The invention also provides a cell comprising the peptide or peptidomimetic, a nucleic acid encoding the amino acid sequence of the peptide or peptidomimetic, and an antibody that binds to the peptide or peptidomimetic.

The invention further provides a method of inhibiting IGF1R activity in a cell comprising introducing a peptide or peptidomimetic described herein into the cell, whereby the activity of IGF1R is inhibited.

The invention also provides a method for inhibiting the growth or proliferation of a cancer cell comprising administering a peptide or peptidomimetic described herein to the cancer cell, whereupon the growth or proliferation of the cancer cell is inhibited.

In addition, the invention provides a method of treating or preventing a disease mediated by IGF1R in a host comprising administering to the host a peptide or peptidomimetic described herein or nucleic acid encoding same, whereby the disease is treated or prevented.

Related compounds, compositions, and methods also are provided, as will be apparent from the detailed description of the invention.

### DESCRIPTION OF THE DRAWINGS

FIG. 1 is a graph showing the percent survival of cancer cell lines after treatment with a peptide-based IGF1R inhibitor at various concentrations.

FIG. 2 is a graph showing the number of breast cancer cells surviving after treatment with different concentrations of peptide-based IGF1R inhibitors in the presence of exogenous IGF-1, expressed as a percentage of untreated cells.

FIG. 3 is a graph showing AKT activity as units of fluorescence in unstimulated cells, cells stimulated with IGF1, and in cells stimulated in IGF1 treated with an IGF1R inhibitor.

### DETAILED DESCRIPTION OF THE INVENTION

The invention provides a peptide or peptidomimetic that inhibits IGF1R activity. For the purposes of this invention, a peptide or peptidomimetic is considered to inhibit IGF1R activity if it inhibits any biological function of IGF1R. The biological functions of IGF1R include, for example, the binding of IGF1R to any of its ligands (e.g., IGF-I or IGF-2) and IGF1R's signal transduction or kinase activity. Thus, for instance, a peptide or peptidomimetic is considered to inhibit IGF1R activity if, in the presence of the peptide or peptidomimetic, IGF1R binding to at least one of its ligands (e.g., IGF-I or IGF-II) is reduced to any degree as compared to the binding of IGF1R to the same ligand in the absence of the peptide or peptidomimetic. A peptide or peptidomimetic also is considered to inhibit IGF1R activity if, in the presence of the peptide or peptidomimetic, the signal transduction or kinase activity of IGF1R is reduced to any degree, regardless of whether there is any reduction in binding between IGF1R and its ligands. Preferably, the peptide or peptidomimetic inhibits IGF1R activity to a degree sufficient to reduce the rate of cell growth of a cancer cell, and/or induce cell death of a cancer cell. Suitable assays to test for such a reduction in the biological activity of IGF1R are known in the art, including binding affinity assays, cell growth and cytotoxicity assays, and gene regulation assays (e.g., luciferase reporter assay).

The invention is not limited with respect to any particular mechanism of action. The peptide or peptidomimetic may inhibit IGF1R by binding to IGF1R or its targets, thereby interfering with IGFR1-ligand binding. Alternatively, or in addition, the peptide or peptidomimetic may inhibit the dimerization of IGF1R. Of course, the peptide or peptidomimetic may act by some other mechanism.

At least a portion of the amino acid sequence of the peptide or peptidomimetic is derived from or based upon the amino acid sequence of the juxtamembrane region of IFG1R. The following are partial sequences of IGF1Rs from several different animals, wherein the juxtamembrane regions of the protein are underlined:

| | | SEQ ID |
|---|---|---|
| Human | IALPVAVLLIVGGLVIMLYVFHRKRNNSRLGNGVLYASVNPEYFSAADVY | 49 |
| Mouse | IALPVAILLIVGGLVIMLYVFHRKRNNSRLGNGVLYASVNPEYFSAADVY | 50 |
| Rat | IALPVAILLIVGGLVIMLYVFHRKRNNSRLGNGVLYASVNPEYFSAADVY | 51 |
| Rat(2) | IALPVAILLIVGGLVIMLYVFHRKRNNSRLGNGVLYASVNPEYFSAADVY | 52 |
| Frog | VAIPLALSFLLVGIISIVCFVFKKRNSNRLGNGVLYASVNPEYFSAAEMY | 53 |
| Zebra- | IIIPVIVLLLLLFVIVAVIIVTKKRNSDRIGNGVLYASVNPEYFSPFEMY | 54 |
| fish | | |
| | : :*: : ::: :: : .. :***..*:***************. ::* | |

Positions in the juxtamembrane region of the sequence that are conserved are marked with an asterisk. Positions of the juxtamembrane region of the sequences showing variation are marked with dots indicating the relative similarity between the residues that occupy a given position in the sequence. Two dots below a given position in the sequence indicate substitution by more closely related residues and one dot or no dots indicates substitution by less similar residues.

The above alignment of IGF1R sequences shows that most of the juxtamembrane region of IGF1R is conserved between species. However, an alignment of the juxtamembrane region of IGF1R with the juxtamembrane region of insulin receptor (IR) illustrates significant dissimilarity:

| | | SEQ ID |
|---|---|---|
| IGF1R | IALPVAVLLIVGGLVIMLYVFHRKRNNSRLGNGVLYASVNPEYFSAADV | 49 |
| IR | IIGPLIFVFLFSVVIGSIYLFLRKRQPDGP-LGPLYASSNPEYLSASDV | 56 |
| | * *: .:::.. :: :*:* ***: . * **** ****:**:** | |

Given the dissimilarity in the sequences, peptide-based inhibitors targeting the juxtamembrane region of IGF1R can selectively inhibit IGF1R over IR. Thus, according to a preferred aspect of the invention, the peptide or peptidomimetic of the invention inhibits IGF1R activity to a greater extent than the peptide or peptidomimetic inhibits IR activity (e.g., inhibits IGF1R activity by 2× or more, 4× or more, 10× or more, 100× or more, or even 1000× or more, as compared to the inhibition of IR activity).

According to one aspect of the invention, the peptide or peptidomimetic comprises the amino acid sequence RXGNGV (SEQ ID NO: 1), which is a motif of the juxtamembrane region of IGF1R. X in the sequence preferably is a non-polar residue, more preferably a hydrophobic or aliphatic residue, such as leucine, isoleucine, or valine.

A peptide or peptidomimetic comprising the amino acid sequence RXGNGV (SEQ ID NO: 1) can further comprise one or more flanking residues. By way of illustration, SEQ ID NOs: 2-23 and 62-68 comprise the amino acid sequence RXGNGV (SEQ ID NO: 1) and one or more flanking residues (Table 1). The flanking residues should be chosen so as not to interfere with the ability of the peptide to inhibit IGF1R activity. Guidance for the selection of such residues is provided by the relevant sequence of the juxtamembrane region of IGF1R itself. For instance, one can choose residues for use in the peptide that are identical to, or have properties similar to, the residues at the corresponding positions of the juxtamembrane region of a given IGF1R (preferably human IGF1R). Preferably, the amino acid used at any given “X” residue of SEQ ID NO: 2-23 and 62-68 has one or more of the properties of columns (A)-(E) of Table 2 for that residue. More preferably the selected amino acid residue has more than one of such indicated properties or even all such indicated properties. By way of further illustration, Table 2 provides exemplary amino acid residues to be used at each position “X” of SEQ ID NOs: 2-23 and 62-68, wherein the most preferred residues are underlined. Of course, other amino acid residues, including synthetic amino acid residues, can be used instead of the exemplary residues, which are provided only for illustration. Specific examples of amino acid sequences comprising the amino acid sequence RXGNGV (SEQ ID NO: 1) include SEQ ID NOs: 24-46 and 57-61.

TABLE 1

| SEQ | |
|---|---|
| ID | |
| NO. | Peptide Sequence |
| 62 | RXGNGVX₁ |
| 2 | RXGNGVX₁X₂ |
| 63 | RXGNGVX₁X₂X₃ |
| 64 | RXGNGVX₁X₂X₃X₄ |
| 3 | RXGNGVX₁X₂X₃X₄X₅ |
| 4 | RXGNGVX₁X₂X₃X₄X₅X₆ |
| 5 | RXGNGVX₁X₂X₃X₄X₅X₆X₇ |
| 6 | X_{1′}RXGNGV |
| 7 | X_{2′}X_{1′}RXGNGV |
| 8 | X_{3′}X_{2′}X_{1′}RXGNGV |
| 9 | X_{4′}X_{3′}X_{2′}X_{1′}RXGNGV |
| 10 | X_{5′}X_{4′}X_{3′}X_{2′}X_{1′}RXGNGV |
| 11 | X_{7′}X_{6′}X_{5′}X_{4′}X_{3′}X_{2′}X_{1′}RXGNGV |
| 12 | X_{8′}X_{7′}X_{6′}X_{5′}X_{4′}X_{3′}X_{2′}X_{1′}RXGNGV |
| 13 | X_{9′}X_{8′}X_{7′}X_{6′}X_{5′}X_{4′}X_{3′}X_{2′}X_{1′}RXGNGV |
| 14 | X_{1′}RXGNGVX₁X₂ |
| 15 | X_{2′}X_{1′}RXGNGVX₁X₂ |
| 16 | X_{3′}X_{2′}X_{1′}RXGNGVX₁X₂ |
| 65 | X_{4′}X_{3′}X_{2′}X_{1′}RXGNGVX₁ |
| 17 | X_{4′}X_{3′}X_{2′}X_{1′}RXGNGVX₁X₂ |
| 66 | X_{4′}X_{3′}X_{2′}X_{1′}RXGNGVX₁X₂X₃ |
| 67 | X_{4′}X_{3′}X_{2′}X_{1′}RXGNGVX₁X₂X₃X₄ |
| 68 | X_{4′}X_{3′}X_{2′}X_{1′}RXGNGVX₁X₂X₃X₄X₅ |
| 18 | X_{4′}X_{3′}X_{2′}X_{1′}RXGNGVX₁X₂X₃X₄X₅X₆ |
| 19 | X_{5′}X_{4′}X_{3′}X_{2′}X_{1′}RXGNGVX₁X₂X₃X₄X₅X₆ |
| 20 | X_{9′}X_{8′}X_{7′}X_{6′}X_{5′}X_{4′}X_{3′}X_{2′}X_{1′}RXGNGVX₁X₂X₃X₄X₅X₆ |
| 21 | X_{8′}X_{7′}X_{6′}X_{5′}X_{4′}X_{3′}X_{2′}X_{1′}RXGNGVX₁X₂X₃X₄X₅ |
| 22 | X_{7′}X_{6′}X_{5′}X_{4′}X_{3′}X_{2′}X_{1′}RXGNGVX₁X₂X₃X₄X₅ |
| 23 | X_{7′}X_{6′}X_{5′}X_{4′}X_{3′}X_{2′}X_{1′}RXGNGVX₁X₂X₃X₄X₅X₆X₇ |

Most preferably, X_{3′}, X_{4′}, and X_{5′}, to the extent they are present in the sequence, are selected to be N, R, and K respectively. Similarly, X_{6′} is preferably chosen to be K or, more preferably, R, to the extent the sequence comprises an amino acid at position X_{6′}.

According to another aspect of the invention, the peptide or peptidomimetic comprises the juxtamembrane region of IGF1R (e.g., human IGF1R, SEQ ID NO: 43), or a fragment thereof comprising about six or more contiguous amino acids, preferably about eight or more contiguous amino acids, or even about ten or more contiguous amino acids. By way of illustration, the fragment of IGF1R can comprise any of SEQ ID NOs: 24-46 or 57-61, which include the previously discussed motif RXGNGV (SEQ ID NO: 1). However, the fragment need not comprise this motif. For example, the fragment can comprise a sequence such as SEQ ID NOs: 47 or 48.

TABLE 2

| | Preferred Amino Acid | | | | | |
|---|---|---|---|---|---|---|
| | | (B) | | | (E) | (F) |
| | (A) | Hydro- | (C) | (D) | Aliphatic/ | Exem- |
| | Polarity | phobic* | Charge | Size** | Aromatic | plary^{†} |
| X₁ | Non-Polar | Hydrophobic | Neutral | — | Aliphatic | L, I, V |
| X₂ | Polar | — | Neutral | — | Aromatic | Y, F, T |
| X₃ | Non-Polar | Hydrophobic | Neutral | Small | — | A |
| X₄ | Polar | — | Neutral | Small | — | S, T |
| X₅ | Non-Polar | Hydrophobic | Neutral | Small | Aliphatic | V, I, L |
| X₆ | Polar | — | Neutral | Small | — | N, D |
| X₇ | Non-Polar | Hydrophobic | Neutral | Small | — | P |
| X_{1′} | Polar | — | Neutral | Small | — | D, N, S |
| X_{2′} | Polar | — | Neutral | Small | — | S, N, T, D |
| X_{3′} | Polar | — | Neutral | Small | — | N, D |
| X_{4′} | Polar | — | Basic | — | — | R, K |
| X_{5′} | Polar | — | Basic | — | — | K, R |
| X_{6′} | Polar | — | Basic | — | — | R, K |
| X_{7′} | Polar | — | Basic | — | Aromatic | H, F, T |
| X_{8′} | Non-Polar | Hydrophobic | Neutral | — | Aromatic | V, F, I |
| X_{9′} | Non-Polar | Hydrophobic | Neutral | Small | Aliphatic | V, F, I |

*Hydropathy index greater than zero. Kyte, J.; Doolittle, R. F., J. Mol. Biol., 157 (1), 105-132 (1982).
**Molecular mass of about 133 or less.
^{†}Underlining indicates preferred residues.

Variant sequences other than those specifically mentioned herein are contemplated, which comprise significant sequence identity (e.g., 80%, 85%, 90%, 95%, 98%, or 99% sequence identity) to the amino acid sequence of the juxtamembrane region of IGF1R (e.g., SEQ ID NO: 43) or fragment thereof comprising at least six or at least eight contiguous amino acids, provided that such variants retain the ability to inhibit IGF1R activity. Such variants can comprise one or more amino acid substitutions, deletions, or insertions as compared to the parent amino acid sequence. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc. Desirably, the peptide or peptidomimetic comprises one or more of SEQ ID NOs 24-48 or 57-61, or a variant thereof comprising up to five substitutions or deletions (e.g., one, two, three, or four substitutions or deletions).

The peptide or peptidomimetic also can comprise synthetic, non-naturally occurring amino acids. Such synthetic amino acids include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The properties of such synthetic amino acids are well-documented. Any natural amino acid of one or more of the sequences discussed herein can be substituted with a synthetic amino acid having similar properties.

The term “peptidomimetic” as used herein refers to a compound that comprises the same general structure of a corresponding polypeptide, but which includes modifications that increase its stability or biological function. For instance, the peptidomimetic can be a “reverso” analogue of a given peptide, which means that the peptidomimetic comprises the reverse sequence of the peptide. In addition, or instead, the peptidomimetic can comprise one or more amino acids in a “D” configuration (e.g., D-amino acids), providing an “inverso” analogue. Peptidomimetics also include peptoids, wherein the sidechain of each amino acid is appended to the nitrogen atom of the amino acid as opposed to the alpha carbon. Peptoids can, thus, be considered as N-substituted glycines which have repeating units of the general structure of NRCH₂CO and which have the same or substantially the same amino acid sequence as the corresponding polypeptide. In this respect, the peptide or peptidomimetic can comprise any of the sequences described herein in reverse order.

Smaller peptides and peptidomimetics are believed to be advantageous for inhibiting IGF1R function and to facilitate entry into a cell. Thus, the peptide or peptidomimetic preferably comprises about 50 or fewer amino acids, such as about 40 or fewer amino acids, about 35 or fewer amino acids, about 25 or fewer amino acids, or even about 20 or fewer amino acids. Generally, however, the peptide or peptidomimetic will comprise at least about 8 amino acids, such as at least about 10 amino acids, or at least about 15 amino acids.

The peptide or peptidomimetic can comprise, consist essentially of, or consist of, any of foregoing sequences or variants thereof. The peptide or peptidomimetic consists essentially of the foregoing sequences if it does not comprise other elements, such as other amino acid sequences, that prevent the peptide from inhibiting IGF1R activity.

The peptide or peptidomimetic coupled to a cell penetrating motif or other moiety so as to more efficiently facilitate the delivery of the peptide to the interior of a cell, anchor the peptide to the cell membrane of a cell, and/or promote folding of the peptide. Thus, the peptide or peptidomimetic can be provided as part of a composition comprising the peptide and cell penetrating motif or other moiety. Any of various cell penetrating motifs and or other moieties useful for these purposes can be used. By way of illustration, suitable cell penetrating motifs and other relevant moieties (e.g., cell-membrane anchoring moieties) include lipids and fatty acids, peptide transduction domains (e.g., HIV-TAT, HSV Transcription Factor (VP22), and penetratin), and other types of carrier molecules (e.g., Pep-1).

According to one aspect of the invention, the cell penetrating motif or other moiety comprises a fatty acid or lipid molecule. The fatty acid or lipid molecule can be, for example, a palmitoyl group, farnesyl group (e.g., farnesyl diphosphate), a geranylgeranyl group (e.g., geranylgeranyl diphosphate), a phospholipid group, glycophosphatidylinositol, phosphatidylserine, phosphatidylethanolamine, sphingomyelin, phosphatidylcholine, cardiolipin, phosphatidylinositol, phosphatidic acid, lysophosphoglyceride, a cholesterol group, and the like. Preferably, the fatty acid molecule is a C₁ to C₂₄ fatty acid or C₈ to C₁₆ fatty acid. Desirably, the fatty acid comprises three or more, four or more, five or more, six or more, seven or more, eight or more, nine or more or ten or more carbon atoms. Typically, the fatty acid will comprise 22 or fewer, 20 or fewer, 18 or fewer, or 16 or fewer carbon atoms. Specific examples of fatty acids include, without limitation, lauric acid, palmitic acid, myristic acid, stearic acid, oleic acid, linoleic acid, α-linoleic acid, linolenic acid, arachidonic acid, timnodonic acid, docosohexenoic acid, erucic acid, arachidic acid, behenic acid.

The fatty acid or lipid molecule can be attached to any suitable part of the peptide or peptidomimetic. In a preferred embodiment of the invention, the fatty acid or lipid molecule is attached at the amino (N-) terminus, the carboxyl (C-) terminus, or both the N- and C-termini of the peptide or peptidomimetic. Typically, the fatty acid or lipid molecule is attached via an amide or ester linkage. When the fatty acid or lipid molecule is attached at the C-terminus of the polypeptide or peptidomimetic, the fatty acid or lipid molecule preferably is modified, e.g., to include an amino group such as NH₂(CH₂)ₙCOOH or CH₃(CH₂)ₘCH(NH₂)COOH, wherein each of n and m is, independently, 1 to 24, preferably 8 to 16. The fatty acid or lipid residue can advantageously be attached to a terminal lysine in the epsilon (c) position.

According to another aspect of the invention, the cell penetrating motif is a peptide transduction domain (also known as protein transduction domains or PTDs). PTDs typically are fused to the IGF1R-inhibitory peptide or peptidomimetic. Thus, the peptide or peptidomimetic can be a fusion protein comprising the peptide or peptidomimetic and a PTD. Often, the fusion protein is cleaved inside of a cell to remove the cell penetrating motif.

The peptide or peptidomimetic can further comprise linking residues disposed between the amino acid sequence derived from or based upon the juxtamembrane region of IGF1R and the cell penetrating motif or other moiety. Illustrative examples of such linking residues include K, KK, RK, RQ, KQ, RQI, KQI, RQIK, and KQIK.

The peptide or peptidomimetic can be prepared by any method, such as by synthesizing the peptide or peptidomimetic, or by expressing a nucleic acid encoding an appropriate amino acid sequence in a cell and harvesting the peptide from the cell. Of course, a combination of such methods also can be used. Methods of de novo synthesizing peptides and peptidomimetics, and methods of recombinantly producing peptides and peptidomimetics are known in the art (see, e.g., Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994).

The invention also provides a nucleic acid encoding the amino acid sequence of the peptide or peptidomimetic. The nucleic acid can comprise DNA or RNA, and can be single or double stranded. Furthermore, the nucleic acid can comprise nucleotide analogues or derivatives (e.g., inosine or phosphorothioate nucleotides and the like). The nucleic acid can encode the amino acid sequence of the peptide or peptidomimetic alone, or as part of a fusion protein comprising such sequence and a cell penetrating motif, as described herein. The nucleic acid encoding the amino acid sequence of the peptide or peptidomimetic can be provided as part of a construct comprising the nucleic acid and elements that enable delivery of the nucleic acid to a cell, and/or expression of the nucleic acid in a cell. Such elements include, for example, expression vectors and transcription and/or translation sequences. Suitable vectors, transcription/translation sequences, and other elements, as well as methods of preparing such nucleic acids and constructs, are known in the art (e.g., Sambrook et al., supra; and Ausubel et al., supra).

The present invention further provides an antibody to the peptide or peptidomimetic, or an antigen binding fragment or portion thereof (e.g., Fab, F(ab′)₂, dsFv, sFv, diabodies, and triabodies). The antibody can be monoclonal or polyclonal, and of any isotype, e.g., IgA, IgD, IgE, IgG, IgM, etc. The antibody can be a naturally-occurring antibody, e.g., an antibody isolated and/or purified from a mammal, e.g., mouse, rabbit, goat, horse, chicken, hamster, human, etc. Alternatively, the antibody can be a synthetic or genetically-engineered antibody, e.g., a humanized antibody or a chimeric antibody. The antibody can be in monomeric or polymeric form. The antibody, or antigen binding portion thereof, can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), or element particles (e.g., gold particles). Such antibodies can be used for any purpose, such as to facilitate the detection or purification of a peptide or peptidomimetic described herein. Suitable methods of making antibodies are known in the art, including standard hybridoma methods, EBV-hybridoma methods, bacteriophage vector expression systems, and phage-display systems (see, e.g., Köhler and Milstein, Eur. J. Immunol., 5, 511-519 (1976); Harlow and Lane (eds.), Antibodies: A Laboratory Manual, CSH Press (1988); C. A. Janeway et al. (eds.), Immunobiology, 5^{th} Ed., Garland Publishing, New York, N.Y. (2001); Haskard and Archer, J. Immunol. Methods, 74(2), 361-67 (1984); Roder et al., Methods Enzymol., 121, 140-67 (1986); Huse et al., Science, 246, 1275-81 (1989); Sambrook et al., supra; Ausubel et al., supra; Knappik et al., J. Mol. Biol. 296: 57-86 (2000)).

The peptide or peptidomimetic, nucleic acid, or antibody can be isolated. The term “isolated” as used herein encompasses compounds or compositions that have been removed from a biological environment (e.g., a cell, tissue, culture medium, body fluid, etc.), or otherwise increased in purity to any degree (e.g., isolated from a synthesis medium). Isolated compounds and compositions, thus, can be synthetic or naturally produced.

A cell comprising the peptide or peptidomimetic, or nucleic acid encoding the amino acid sequence of the peptide or peptidomimetic, also is provided herein. Such a cell includes, for example, a cell engineered to express a nucleic acid encoding the amino acid sequence of the peptide or peptidomimetic. Suitable cells include prokaryotic and eukaryotic cells, e.g., mammalian cells, yeast, fungi, and bacteria (such as E. coli). The cell can be in vitro, as is useful for research or for production of the peptide or peptidomimetic, or the cell can be in vivo, for example, in a transgenic mammal that expresses the peptide.

The peptide or peptidomimetic can be used for any purpose, but is especially useful for inhibiting IGF1R activity in a cell. Thus, provided herein is a method of inhibiting IGF1R activity in a cell, which method comprises administering a peptide or peptidomimetic described herein to a cell in an amount sufficient to inhibit IGF1R activity.

The peptide or peptidomimetic can be administered to the cell by any method. For example, the peptide or peptidomimetic can be administered to a cell by contacting the cell with the peptide or peptidomimetic, typically in conjunction with a regent or other technique (e.g., microinjection or electroporation) that facilitates cellular uptake. Alternatively, and preferably, the peptide or peptidomimetic is administered by contacting the cell with a composition comprising the peptide or peptidomimetic and a cell penetrating motif, as discussed herein.

The peptide or peptidomimetic also can be administered by introducing a nucleic acid encoding the amino acid sequence of the peptide into the cell such that the cell expresses a peptide comprising the amino acid sequence. The nucleic acid encoding the peptide can be introduced into the cell by any of various techniques, such as by contacting the cell with the nucleic acid or a composition comprising the nucleic acid as part of a construct, as described herein, that enables the delivery and expression of the nucleic acid. Specific protocols for introducing and expressing nucleic acids in cells are known in the art (see, e.g., Sambrook et al. (eds.), supra; and Ausubel et al., supra).

The peptide, peptidomimetic, or nucleic acid can be administered to a cell in vivo by administering the peptide, peptidomimetic, or nucleic acid comprising the cell. The host can be any host, such as a mammal, preferably a human. Suitable methods of administering peptides, peptidomimetics, and nucleic acids to hosts are known in the art, and discussed in greater detail in connection with the pharmaceutical composition comprising such compounds, below.

The cell can be any type of cell that comprises IGF1R. Preferably, the cell is of a type that is related to a disease or condition mediated by IGF1R activity. For example, the cell can be an engineered cell that is designed to mimic a condition or disease associated with IGF1R activity, or the cell can be a cell of a patient afflicted with a disease or condition associated with IGF1R activity. Diseases mediated by IGF1R include diseases characterized by IGF1R overexpression or overactivity, or diseases characterized by the overexpression or overactivity of any of IGF1R ligands (e.g., IGF1 or IGF2). Cancer cells are one example of a cell type that can be used. The cell can be in vitro or in vivo in any type of animal, such as a mammal, preferably a human.

The method of inhibiting IGF1R activity in a cell can be used for any purpose, such as for the research, treatment, or prevention of diseases or conditions mediated by IGF1R. IGF1R activity has been linked to a large variety of cancers. Thus, according to one aspect of the method of the invention, the peptide or peptidomimetic is administered to a cancer cell, in vitro or in vivo, and administration of the peptide or peptidomimetic to the cancer cell inhibits the growth or survival of the cancer cell.

The cancer cell can be a cell of any type of cancer, in vitro or in vivo, particularly those associated with IGF1R activity, such as those associated with IGF1 or IGFII overexpression or up-regulation of IGF1R. Non-limiting examples of specific types of cancers include cancer of the head and neck, eye, skin, mouth, throat, esophagus, chest, bone, lung, colon, sigmoid, rectum, stomach, prostate, breast, ovaries, kidney, liver, pancreas, brain, intestine, heart or adrenals. More particularly, cancers include solid tumor, sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, retinoblastoma, a blood-born tumor, acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acutenonlymphocyctic leukemia, acute undifferentiated leukemia, chronic myelocytic leukemia, chronic lymphocytic leukemia, hairy cell leukemia, or multiple myeloma. See, e.g., Harrison's Principles of Internal Medicine, Eugene Braunwald et al., eds., pp. 491 762 (15th ed. 2001). The methods of the invention are believed to be especially useful for the treatment of sarcomas (e.g., osteosarcoma and rhabdomyosarcoma), breast, prostate, colon, lung, pancreatic, cervical, ovarian, and endometrial cancers, melanoma, neuroblastoma, multiple myeloma, and hepatocellular carcinoma, as well as any other cancer known to be responsive to IGF1R inhibitors.

IGF1R activity also has been linked to other diseases, including benign prostatic hyperplasia (BPH), diarrhea associated with metastatic carcinoid and vasoactive intestinal peptide secreting tumors (e.g., VIPoma or Werner-Morrison syndrome), acromegaly, gigantism, psoriasis, atherosclerosis, spinocerebellar ataxia and smooth muscle restenosis of blood vessels or inappropriate microvascular proliferation, such as that found as a complication of diabetes, especially of the eye. Thus, the methods of the invention are believed to be useful for the treatment of such diseases as well.

Peptides and peptidomimetics, as described herein, include salts, esters, alkylated (e.g., methylated), and acetylated peptides. Any one or more of the compounds or compositions of the invention described herein (e.g., peptide or peptidomimetic, nucleic acid, antibody, or cell) can be formulated as a pharmaceutical composition, comprising a compound of the invention and a pharmaceutically acceptable carrier. Furthermore, the compounds or compositions of the invention can be used in the methods described herein alone or as part of a pharmaceutical formulation.

The pharmaceutical composition can comprise more than one compound or composition of the invention. Alternatively, or in addition, the pharmaceutical composition can comprise one or more other pharmaceutically active agents or drugs. Examples of such other pharmaceutically active agents or drugs that may be suitable for use in the pharmaceutical composition include anticancer agents. Suitable anticancer agents include, without limitation, alkylating agents; nitrogen mustards; folate antagonists; purine antagonists; pyrimidine antagoinists; spindle poisons; topoisomerase inhibitors; apoptosis inducing agents; angiogenesis inhibitors; podophyllotoxins; nitrosoureas; cisplatin; carboplatin; interferon; asparginase; tamoxifen; leuprolide; flutamide; megestrol; mitomycin; bleomycin; doxorubicin; irinotecan; and taxol, geldanamycin (e.g., 17-AAG), and various anti-cancer peptides and antibodies.

The carrier can be any of those conventionally used and is limited only by physio-chemical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular compound or composition of the invention and other active agents or drugs used, as well as by the particular method used to administer the compound and/or inhibitor. Accordingly, there are a variety of suitable formulations of the pharmaceutical composition of the present inventive methods. The following formulations for oral, aerosol, parenteral, subcutaneous, intravenous, intramuscular, interperitoneal, rectal, and vaginal administration are exemplary and are in no way limiting. One skilled in the art will appreciate that these routes of administering the compound of the invention are known, and, although more than one route can be used to administer a particular compound, a particular route can provide a more immediate and more effective response than another route.

Injectable formulations are among those formulations that are preferred in accordance with the present invention. The requirements for effective pharmaceutical carriers for injectable compositions are well-known to those of ordinary skill in the art (See, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed., pages 622-630 (1986)).

Topical formulations are well-known to those of skill in the art. Such formulations are particularly suitable in the context of the present invention for application to the skin.

Formulations suitable for oral administration can consist of (a) liquid solutions, such as an effective amount of the inhibitor dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The compounds and compositions of the invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compounds and compositions of the invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Additionally, the compounds of the invention, or compositions comprising such compounds, can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

The following examples further illustrate the invention but, of course, should not be construed as in any way limiting its scope.

### EXAMPLES

The following techniques were used in connection with the examples described herein unless expressly stated otherwise.

Peptide Synthesis and Purification: Peptides were synthesized on a 433A Peptide Synthesizer (Applied Biosystems, Foster City, Calif.) using Fmoc chemistry at 0.1 mmol scale. The peptides were cleaved from the resin and deprotected with a mixture of 90.0% (v/v) trifluoroacetic acid (TFA) with 2.5% water, 2.5% triisopropyl-silane, and 5% thioanisol. The resin and deprotection mixture were pre-chilled to −5° C. (in ice mixed with NaCl) and reacted for 10 minutes at −5° C. with stirring. After 10 minutes, the reaction was allowed to continue on at room temperature for 1 hour and 45 minutes. The resin was filtered off and the product was precipitated with cold diethyl ether. The resin was washed with neat TFA to ensure maximum product yield. Peptide suspended in diethyl ether was centrifuged for ten minutes at −20° C., excess ether decanted. The precipitate was washed with diethyl ether four more times and left to dry in a vacuum overnight. The dried crude peptide was then analyzed by electrospray LC/MS on Agilent 1100 series instrument (Agilent Technologies, Palo Alto, Calif.) with the use of Zorbax 300SB-C18 column (Agilent Technologies, Palo Alto, Calif.). The samples were dissolved in DMSO and purified in a Waters 500 HPLC system on a preparative (25 mm×250 mm) Atlantis C18 reverse phase column (Agilent Technologies, Palo Alto, Calif.) in a 90 minute gradient of 0.1% (v/v) trifluoroacetic acid in water and 0.1% trifluoroacetic acid in acetonitrile, starting with a 10 mL/min flow of water with 0 mL/min flow of acetonitrile moving to a 10 mL/min flow of acetonitrile and a 0 mL/min flow of water. The fractions containing peptides were analyzed on Agilent 1100 LC/MS with the use of a Zorbax 300SB-C3 Poroshell column and a gradient of 5% acetic acid in water and acetonitrile. Fractions that were more than 95% pure were combined and freeze dried. When freeze drying, acetic acid was added to the final concentration of 5% to make sure the peptides were converted into acetate salts. Retro-inverso peptide made of all-D amino acids was synthesized using essentially the same protocol, except that palmitic acid had to be introduced in the side chain.

Cell Toxicity Assays: MCF-7 (breast cancer), T47D (breast cancer), Colo 205 (colon cancer), JM-1 (rat hepatoma), Sk Me1-2 (melanoma), PLC (human hepatoma), and HepG2 (human hepatoma) cells were obtained from American Type Cell Culture Collection. MCF-7 cells were grown in RPMI medium supplemented with 10% Fetal Bovine Serum. The rest of the cell lines were grown in DMEM medium supplemented with 10% Fetal Bovine Serum.

MTT 43-(4,5-Dimethylthiazol-2-yl)-2,5-diphenyltetrazolium) cell assay was performed using MCF-7 breast cancer cells. For the assay, cells were seeded into 96 well plates in medium containing 1% Fetal Bovine Serum and 100 μL of a cell suspension containing 5000 cells per well. Wells containing medium-only, and wells containing untreated cells, served as controls.

Seeded cells were allowed to attach and grow in the wells for 24 hours. A 5 mM stock solution of each peptide was made by dissolving approximately 1 mg of peptide in appropriate amount of DMSO. 2 mL dilutions were used to make 0.2 μM, 1.0 μM, 5.0 μM, and 10.0 μM concentration solutions of the compounds in serum-free medium. 100 μl, of the resulting 2× solutions were added to the corresponding wells to provide a final FBS concentration of 0.5% and 0.1 μM, 0.5 μM, 2.5 μM, and 5.0 μM dilutions of inhibitors. The cells were incubated with test compounds for two days. The control wells were given 100 μL of medium to ensure maximum similarity to the test wells so that all of the wells had 200 μL of medium during the growth period. An individual plate was made for Time Zero, to which the dye was added at the time of addition of compounds.

An alternative potency assay was used to test compounds in MCF-7 (breast cancer), T47D (breast cancer), Colo 205 (colon cancer), JM-1 (rat hepatoma), Sk MeI-2 (melanoma), PLC (human hepatoma), and HepG2 (human hepatoma) cells. Cells were suspended at a concentration of approximately 5000 cells per 100 μL of suspension in a 5% FBS RPMI medium. The cells were seeded into 96-count wells and incubated over night. After one day's incubation, the 5% FBS medium was aspirated and replaced with 100 μL of no serum RPMI medium containing 1 mg Bovine Serum Albumin (BSA) per 1 mL RPMI. Various concentrations of the test compounds were prepared by dilution in no-serum medium containing 1 mg BSA/mL. 75 μL of the test compound solutions were added to the wells, and the cells were allowed to incubate for 15-20 minutes, after which time 25 μL of human recombinant IGF-1 (Peprotech) solution was added to the wells to attain final concentrations of 10 ng IGF-1/mL medium. The control wells were given 175 μL of no-serum medium in addition to 25 μL of IGF-1 solution. An individual plate was made for time zero, in which the medium was also aspirated and replaced with 175 μL of no-serum medium and 25 μL of IGF-1. 15 μL of MTT dye was added and let incubate for 4 hours, after which time a stop stop solubilization solution was added. The stop solution lysed the cells and released the dye reduced by the remaining living cells. Absorbance of the wells at 544 nm was determined by a FLUOstar/POLARstar Galaxy (BMG Lab Technologies GmbH) microplate reader. The activity was calculated from the data using the formula: 100×[(T−T₀)/(C−T₀)] for T>T₀ and 100×[(T−T₀)/T₀] for T<T₀, wherein T₀ corresponds to cell density at the time of drug addition, “T” is the optical density of the test well after a 48-h period of exposure to test drug, and “C” is the control optical density. The GI₅₀ measures the growth inhibitory power of the test agent, and is defined as the concentration of test drug where 100×(T−T₀)/(C−T₀)=50. The TGI is the concentration of test drug where 100×(T−T₀)/(C−T₀)=0. Thus, the TGI signifies a cytostatic effect.

AKT Kinase Assay: OMNIA™ fluorogenic AKT Kinase Activity Assay Kit (BioSource through Invitrogen) was used for the measurement of AKT activity in cell lysates. To prepare for the cell treatment, MCF-7 cells were seeded into four wells on a six-well plate in RPMI medium. The medium was supplemented with 10% Fetal Bovine Serum to aid the cell attachment. The cells were incubated for 24 hours, after which time the 10% serum medium was aspirated and replaced with 2 mL of no-serum medium. The cells were starved overnight to stop the signaling from the FBS. On the third day, the medium was replaced and different dilutions of peptide diluted in no-serum medium was added to the four wells. Well 1 & 2 had 0.0 μM of 18, well 3 0.5 and well 4 2.5 μM. One hour after the addition of the compounds, cells were stimulated with 25 ng IGF-1R/mL for 20 min as per the suggested time for IGF-1 to activate AKT(26).

The cells were placed on ice briefly to aid in detaching and then scraped from the bottom of the wells and rinsed with cold PBS. The cells suspended in PBS were collected and centrifuged for 5 minutes at 1500 rpm at 4° C. and the medium aspirated. The cells were lysed with 5Xs the volume of the cell pellet with OMNIA™ Cell Extraction Buffer (Biosource, Kinase Activity Assay Kit, Catalog #KNZ0011). Cell lysis buffer was supplemented with 254 of 100× protease inhibitor cocktail (Sigma-Aldrich) and 254 of a 100× phosphotase inhibitor cocktail (Sigma-Aldrich) per 2.5 mL mixture. The cells were briefly sonicated to break up the DNA and cell parts, then centrifuged at 13000 rpm and 4° C. for 10 min to remove cell debris creating a clear cell extract.

Master mix AKT fluorogenic substrate was prepared according to the protocol provided by BioSource. 10 μl of each cell extract were placed in a white 96-well plate. 50 μL of the master mix were added to each well containing extracts and the measurement of the kinetics of the reaction was started immediately. The fluorescence was excited at 360 nm and read at 485 nm for a length of 60 minutes every thirty seconds on a FLUOstar/POLARstar Galaxy (BMG Lab Technologies GmbH) microplate reader.

Circular Dichroism Spectroscopy: Spectral analysis was performed on 0.5 mg/ml solutions of peptides in deionized water. Peptides were dissolved in water because solubility in phosphate buffer was very low. Since peptide powders were prepared as acetate salts, the pH of the water solutions was in the acidic range, approximately 4.2 for all peptides tested. All attempts to adjust pH to neutrality caused the precipitation of the peptides. UV spectra were used to determine the exact concentration of peptides in solutions. The spectra were taken in 1 cm quartz cuvettes on a HP UV spectrometer (Agilent Technologies, Palo Alto, Calif.). Extinction coefficients at 280 nm were determined with the use of ProtParam software (http://expasy.org/tools/protparam.html). The solutions were transferred to 1 mm cuvettes for analysis on the Circular Dichroism Spectrometer Model 202 (AVIV Instruments Inc.). The data was acquired and processed using CDs Software (AVIV BioMedical, Inc. Lakewood, N.J.).

### Example 1

The following example illustrates the use of peptides and peptidomimetics to inhibit IGF1R activity in breast cancer cells, in accordance with the invention. Four peptides were synthesized, which corresponded to the entire IGF1R juxtamembrane (JM) region (residues 929-954) (peptide 1) and truncated versions of the JM region (peptides 1-3) (see Table 3). The truncations were introduced at Gly and Pro residues believed to cause bends in the secondary structures of proteins and peptides. Several additional peptides were prepared (peptides 5-24 and 27) by further truncation or elongation of peptides 1-4. To enable cell penetration, all peptides were equipped with palmitoyl residue on the N-terminus. The palmitoyl residue also anchors the JM analogs to the plasma membrane, thus providing for a better mimic of the native structure of the JM region. The ability of the cells to inhibit IGF1R resulting in inhibition of cell growth or survival was evaluated in MCF-7 breast cancer cells using the above-described MTT assay. The results are presented in Table 3.

All of the peptides showed growth inhibition in MCF-7 breast cancer cells, which are known to be IGF1R dependent. Each of peptides 1-4 showed mild growth inhibition, whereas peptide 3 also exhibited cell killing activity. Truncation of peptide 3 by one residue (peptide 5) increased the potency, as did addition of Pro to the C-terminus (peptide 23). However, further C-terminal truncation of peptide 5 resulted in gradual decrease in growth inhibitory activity (peptides 7-11). Elimination of the N-terminal H is of peptide 3 decreased activity slightly (peptide 12), but further removal of the N-terminal Arg (peptide 13) and Lys (peptide 14) improved potency more than ten-fold with further truncation having little effect on activity. The most potent derivative, peptide 16, was obtained by combining N-terminal and C-terminal truncations used in peptides 9 and 14. Further shortening of peptide 16 resulted in gradual reduction in potency (peptides 15, 17, 19, 20).

The sequence of peptide 6 was used for the design of homologous peptide corresponding to a part of JM region of the Insulin receptor, 1R-1. Growth-inhibitory properties IR-1 are not surprising because insulin is known to stimulate the growth of breast cancer cells. IFG1R and insulin receptors form functional heterodimers. Thus, inhibition of either of receptors may influence the activity of the dimers. In fact, the IR-1 based peptide inhibited growth of the breast cancer cells.

In order to obtain a metabolically more stable inhibitor of IGF1R, a retro-inverso version of peptide 27 was prepared (peptide 18). A palmitic acid residue was introduced to the side chain of the C-terminal Lys as previously described in Remsberg et al., J. Med. Chem., 50, 4534-4538 (2007). Inversion of the sequence with simultaneous change of all residues into D-amino acids increased the potency of the inhibitor. Since circular dichroism spectroscopy studies were consistent with peptide 18 adopting a O-hairpin conformation, another retro-inverso peptide was generated with the palmitic acid residue on the other terminus of the peptide. Palmitoylation on the N-terminus is much easier and less expensive than that on the C-terminus. The resulting peptide 22 was equipotent to peptide 18 in cell growth inhibition and even demonstrated enhanced cell killing activity. Extension of peptide 22 on the N-terminus (peptide 21) did not improve or diminish the inhibitory activity. Peptide 22 (which is the retro-inverso of peptide 16 with all D-amino acids) showed a larger degree of folding in lipid micelles than all-L peptide 16, which is believed to contribute to higher inhibitory activity.

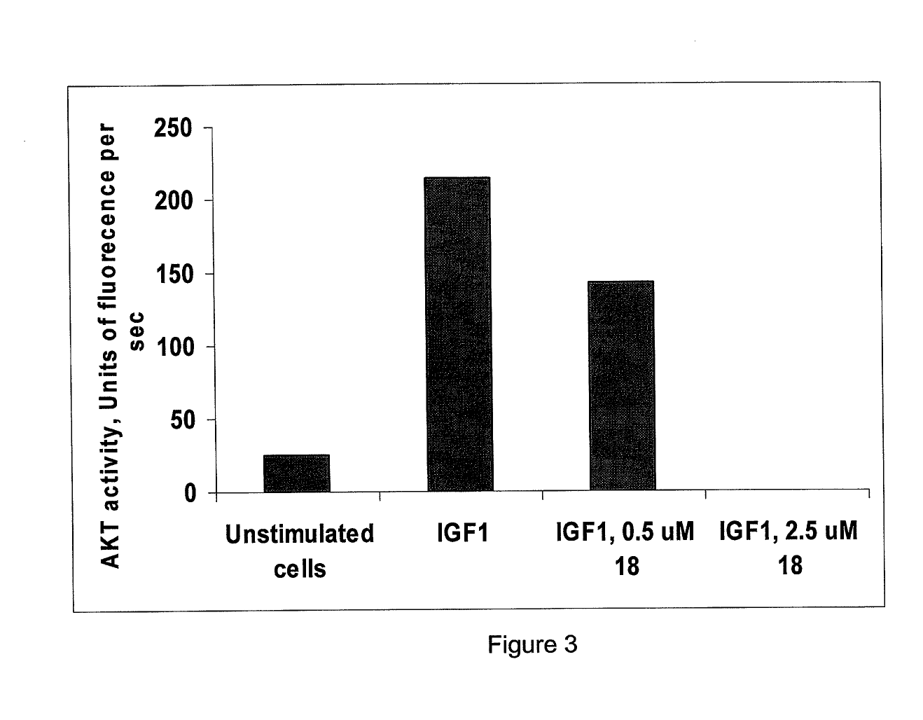

Conformation stability can be an important contributor to biological activity of short peptides, which tend to be flexible and unstructured in solutions. We used Circular Dichroism (CD) spectroscopy to evaluate the conformation and degree of ordering in IGF-1R inhibitors. The spectra were collected in unbuffered water at pH 4.5. The peptides tended to adopt a β-type conformation in solution (FIG. 3) in agreement with the x-ray structure of IGF1R (Favelyukis et al., Nat. Struct. Biol., 8, 1058-1063 (2001); Munshi et al., Acta Crystallogr. D. Biol. Crystallogr., 59, 1725-1730 (2003)). The extent of ordering differed significantly for different peptides. The lowest degree of ordering characterized by the minimum around 200 nm was observed in peptide 3, which was the longest among tested peptides. Truncation of 3 by two residues from the N-terminus resulted in significant increase in O-type structure (peptide 13) and 12.5-fold increase in toxicity. Truncation of 4 residues from the C-terminus (peptide 9) did not improve the degree of folding or the potency. The most potent of the regular all-L peptides, peptide 16, had the extent of folding that was second only to its slightly truncated version, peptide 15. It also has been observed that non-palmytoilated peptide (28) was unordered in water and membrane-mimicking micelles, whereas palmytoilated peptide (16) was folded in both water and micelles. Furthermore, experiments showing decreased effect of peptide inhibitors towards cell-free kinase domain of IGF-1R suggest that membrane anchoring of the peptide inhibitor improves effectiveness.

TABLE 3

| Peptide | Peptide Sequence | GI₅₀ (μM) | TGI (μM) |
|---|---|---|---|
| 1 | Pal-HRKRNNSRLGNG-NH₂ | 1.8 ± 0.05 | >5 |
| <SEQ ID NO: 48> | | | |
| 2 | Pal-HRKRNNSRLG-NH₂ | 1.3 ± 0.05 | >5 |
| <SEQ ID NO: 47> | | | |
| 3 | Pal-HRKRNNSRLGNGVLYASVN-NH₂ | 1.0 ± 0.05 | 1.55 ± 0.1 |
| <SEQ ID NO: 42> | | | |
| 4 | Pal-HRKRNNSRLGNGVLYASVNPEYFSAA-NH₂ | 1.0 ± 0.05 | >5 |
| <SEQ ID NO: 43> | | | |
| 23 | Pal-HRKRNNSRLGNGVLYASVNP-NH₂ | 0.1 ± 0.05 | >5 |
| <SEQ ID NO: 41> | | | |
| 5 | Pal-HRKRNNSRLGNGVLYASV-NH₂ | 0.45 ± 0.05 | 1.45 ± 0.3 |
| <SEQ ID NO: 40> | | | |
| 6 | Pal-VHRKRNNSRLGNGVLYASV-NH₂ | 0.25 ± 0.03 | 1.05 ± 0.1 |
| <SEQ ID NO: 39> | | | |
| 7 | Pal-HRKRNNSRLGNGVLYAS-NH₂ | 1.45 ± 0.1 | 4.1 ± 0.3 |
| <SEQ ID NO: 38> | | | |
| 8 | Pal-HRKRNNSRLGNGVLYA-NH₂ | 1.4 ± 0.1 | >5 |
| <SEQ ID NO: 37> | | | |
| 9 | Pal-HRKRNNSRLGNGVLY-NH₂ | 1.65 ± 0.1 | >5 |
| <SEQ ID NO: 36> | | | |
| 10 | Pal-HRKRNNSRLGNGVL-NH₂ | 1.8 ± 0.1 | >5 |
| <SEQ ID NO: 35> | | | |
| 11 | Pal-HRKRNNSRLGNGV-NH₂ | 2.5 ± 0.1 | >5 |
| <SEQ ID NO: 34> | | | |
| 24 | Pal-VFHRKRNNSRLGNGVLYASVN-NH₂ | 0.1 ± 0.05 | 1.1 ± 0.1 |
| <SEQ ID NO: 33> | | | |
| 12 | Pal-RKRNNSRLGNGVLYASVN-NH₂ | 1.2 ± 0.1 | 2.7 ± 0.3 |
| <SEQ ID NO: 32> | | | |
| 13 | Pal-KRNNSRLGNGVLYASVN-NH₂ | 0.08 ± 0.007 | 0.5 ± 0.08 |
| <SEQ ID NO: 31> | | | |
| 14 | Pal-RNNSRLGNGVLYASVN-NH₂ | 0.1 ± 0.004 | 0.4 ± 0.02 |
| <SEQ ID NO: 30> | | | |
| 27 | Pal-KRNNSRLGNGVLY-NH₂ | 1.1 ± 0.06 | 2.5 ± 0.5 |
| <SEQ ID NO: 29> | | | |
| 16 | Pal-RNNSRLGNGVLY-NH₂ | 0.07 ± 0.005 | 0.5 ± 0.02 |
| <SEQ ID NO: 28> | | | |
| 15 | Pal-NNSRLGNGVLY-NH₂ | 0.1 ± 0.01 | 1.5 ± 0.05 |
| <SEQ ID NO: 27> | | | |
| 17 | Pal-NSRLGNGVLY-NH₂ | 0.2 ± 0.01 | 1.5 ± 0.1 |
| <SEQ ID NO: 26> | | | |
| 19 | Pal-SRLGNGVLY-NH₂ | 0.15 ± 0.05 | 2.0 ± 0.1 |
| <SEQ ID NO: 25> | | | |
| 20 | Pal-RLGNGVLY-NH₂ | 0.2 ± 0.006 | >5 |
| <SEQ ID NO: 24> | | | |

| | | | |
|---|---|---|---|
| 18(27) | Ac-YLVGNGLRSNNRK-(ε-Pal)* | 0.04 ± 0.001 | 0.4 ± 0.02 |
| <SEQ ID NO: 44> | | | |

| | | | |
|---|---|---|---|
| 22(16) | Pal-YLVGNGLRSNNR-NH₂* | 0.04 ± 0.001 | 0.5 ± 0.025 |
| <SEQ ID NO: 45> | | | |
| 21(~14) | Pal-VSAYLVGNGLRSNNR-NH₂* | 0.04 ± 0.001 | 0.1 ± 0.005 |
| <SEQ ID NO: 46> | | | |
| IR-2 | Pal-RQPDGPLGPLY-NH₂ | 0.06 ± 0.001 | 0.4 ± 0.05 |
| <SEQ ID NO: 55> | | | |
| 29 | Pal-RNNSRLGNGVLF-NH₂ | 0.1 ± 0.005 | 1.2 ± 0.05 |
| <SEQ ID NO: 57> | | | |
| 30 | Pal-RNNSRLGNGVL-NH₂ | 1.7 ± 0.05 | 3.7 ± 0.05 |
| <SEQ ID NO: 58> | | | |
| 33 | Pal-RNNSRLGNGVLYA-NH₂ | 0.1 ± 0.05 | >5 |
| <SEQ ID NO: 59> | | | |
| 34 | Pal-RNNSRLGNGVLYAS-NH₂ | 0.04 ± 0.001 | >5 |
| <SEQ ID NO: 60> | | | |
| 35 | Pal-RNNSRLGNGVLYASV-NH₂ | 0.05 ± 0.001 | 2.5 ± 0.5 |
| <SEQ ID NO: 61> | | | |

*Peptides are all D-amino acids

Spectrum of retro-inverso peptide 18 was similar to an inverted spectrum of the parent peptide. However, a significantly stronger signal at 215 nm and better defined maximum at 195 nm suggested a higher degree of ordering and stronger presence of β-type structure. Higher degree of folding correlated with higher potency.

### Example 2

The following example illustrates the cell-growth inhibitory properties of peptides of the invention in various cell lines, and shows that growth inhibition is due to inhibition of IGF1R activity.

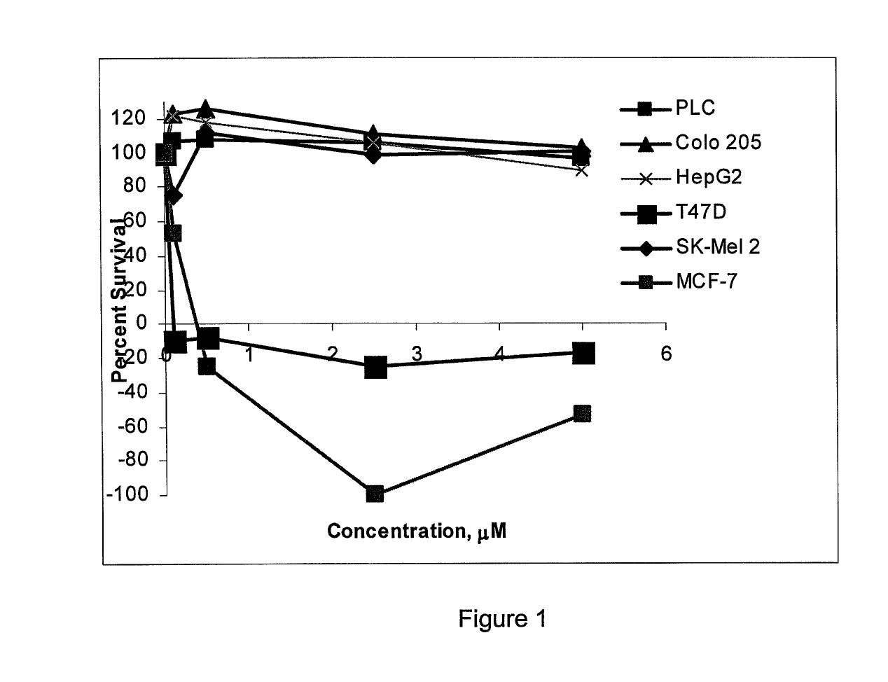

Peptide 9 of Example 1, one of the most potent IGF-1R JM analogs, was tested for cell growth inhibition of MCF-7 (breast cancer), T47D (breast cancer), Colo 205 (colon cancer), JM-1 (rat hepatoma), Sk MeI-2 (melanoma), PLC (human hepatoma), and HepG2 (human hepatoma) cells using the alternative assay described above. The results are presented in FIG. 1. As the results show, peptide 9 exhibited significant cell-growth inhibitory properties in breast cancer cell lines. Lack of activity in some cell lines did not reflect the insensitivity of corresponding tumors because prolonged culturing of tumor cells in medium containing high serum levels selects for cells that are dependent on growth factors present in serum rather than the ones provided by tumor environment.

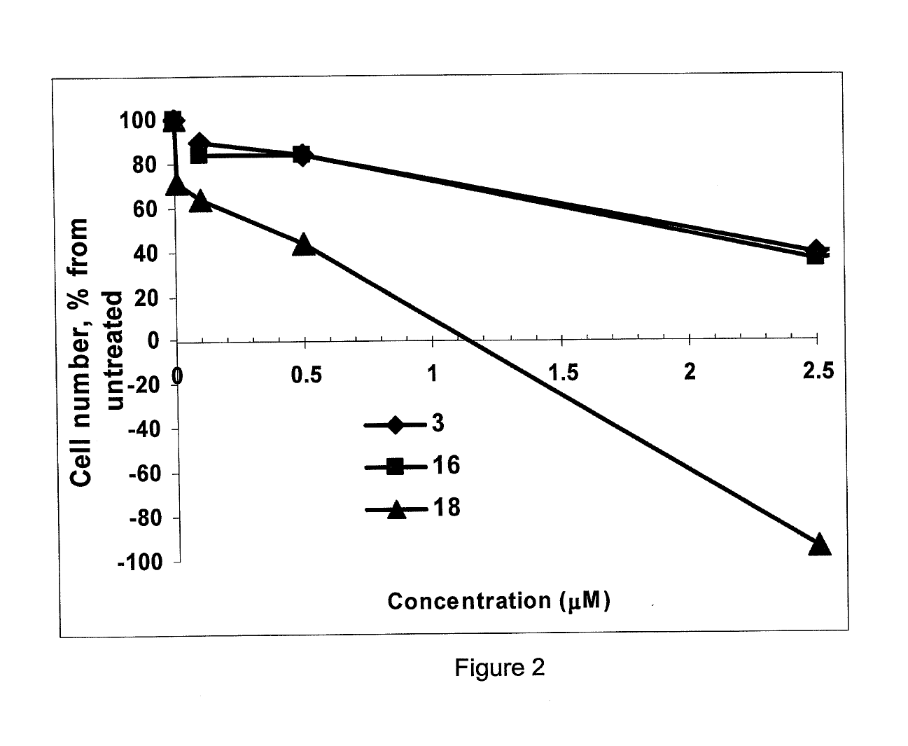

The toxicity tests were conducted in 0.5% serum to minimize the effects of activation of multiple pathways by many growth factors present in bovine fetal serum. To verify that growth inhibitory effects of the peptide JM analogs were due to inhibition of IGF-1-mediated cell growth, the peptide inhibitors were tested on MCF-7 cells grown in serum-free conditions, but in the presence of human recombinant IGF-1 (10 μM/ml). The results are presented in FIG. 2. Significant inhibitory effects in this test confirmed that the observed growth inhibition was due to blocking the IGF-1R-mediated cell growth.

Other experiments have shown that peptide inhibitors described herein are less effective in inhibiting insulin-induced growth of MCF-7 cells indicating that the peptide inhibitors selectively inhibit IGF-1R over insulin receptor.

### Example 3

The following example illustrates that the peptides and peptidomimetics of the invention inhibit IGF-1R kinase activity.

Stimulation of IGF-1 receptor is known to lead to activation of AKT kinase, which is considered to be a marker of IGF pathway signaling. Peptide 18 of Example 1 was tested for the ability to change the levels of intracellular AKT activity in cells stimulated with IGF-1 utilizing a fluorogenic substrate of the kinase. The AKT assay is described above, and the results of the assay are presented in FIG. 3. As expected, the non-stimulated cells exhibited little AKT activation. Addition of IGF-1 lead to significant elevation of enzyme activity. As the concentration of inhibitor increased from 0 to 0.5 μM to 2.5 μM the enzyme activity decreased to undetectable levels.

Other experiments have shown that peptide inhibitors described herein are less effective in inhibiting insulin-induced AKT activity in MCF-7 cells indicating that the peptide inhibitors selectively inhibit IGF-1R over insulin receptor.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A peptide or peptidomimetic comprising the amino acid sequence RXGNGV (SEQ ID NO: 1) or the inverse thereof, wherein the peptide or peptidomimetic comprises about 50 or fewer amino acids and inhibits IGF1R activity.

2. The peptide or peptidomimetic of claim 1, wherein X is a non-polar amino acid.

3. The peptide or peptidomimetic of claim 1, wherein X is a neutral amino acid.

4. The peptide or peptidomimetic of claim 1, wherein X is leucine, isoleucine, or valine.

5. The peptide or peptidomimetic of claim 1 comprising about 25 or fewer amino acids.

6. The peptide or peptidomimetic of claim 1 comprising the amino acid sequence

| | |
|---|---|
| RXGNGVX₁, | (SEQ ID NO: 62) |
| RXGNGVX₁X₂, | (SEQ ID NO: 2) |
| RXGNGVX₁X₂X₃, | (SEQ ID NO: 63) |
| RXGNGVX₁X₂X₃X₄, | (SEQ ID NO: 64) |
| RXGNGVX₁X₂X₃X₄X₅, | (SEQ ID NO: 3) |
| RXGNGVX₁X₂X₃X₄X₅X₆, | (SEQ ID NO: 4) |
| or | |
| RXGNGVX₁X₂X₃X₄X₅X₆X₇, | (SEQ ID NO: 5) |

or the inverse of any such sequences, wherein X₁, X₃, X₅, and X₇ are non-polar amino acids and X₂, X₄, and X₆ are polar amino acids.

7. The peptide or peptidomimetic of claim 4, wherein X₁, X₂, X₃, X₄, X₅, X₆, and X₇ are neutral amino acids.

8. The peptide or peptidomimetic of claim 1 comprising the amino acid sequence

| | |
|---|---|
| X_{1′}RXGNGV, | (SEQ ID NO: 6) |
| X_{2′}X_{1′}RXGNGV, | (SEQ ID NO: 7) |
| X_{3′}X_{2′}X_{1′}RXGNGV, | (SEQ ID NO: 8) |
| X_{4′}X_{3′}X_{2′}X_{1′}RXGNGV | (SEQ ID NO: 9) |
| X^{5′}X^{4′}X_{3′}X_{2′}X_{1′}RXGNGV | (SEQ ID NO: 10) |
| X_{7′}X_{6′}X_{5′}X_{4′}X_{3′}X_{2′}X_{1′}RXGNGV, | (SEQ ID NO: 11) |
| X_{8′}X_{7′}X_{6′}X_{5′}X_{4′}X_{3′}X_{2′}X_{1′}RXGNGV, | (SEQ ID NO: 12) |
| or | |
| X_{9′}X_{8′}X_{7′}X_{6′}X_{5′}X_{4′}X_{3′}X_{2′}X_{1′}RXGNGV, | (SEQ ID NO: 13) |

or the inverse of any such sequences, wherein X_{1′}, X_{2′}, X_{3′}, X_{4′}, X_{5′}, X_{6′} and X_{7′} are polar amino acids, and X_{8′} and X_{9′} are non-polar amino acids.

9. The peptide or peptidomimetic of claim 8, wherein X_{1′}, X_{2′}, X_{3′}, X_{8′} and X_{9′} are neutral amino acids, and X_{4′}, X_{5′}, X_{6′}, and X_{7′} are basic amino acids.

10. The peptide or peptidomimetic of claim 1 comprising the amino acid sequence
(SEQ ID NO: 14)
X_{1′}RXGNGVX₁X₂,
(SEQ ID NO: 15)
X_{2′}X_{1′}RXGNGVX₁X₂,
(SEQ ID NO: 16)
X_{3′}X_{2′}X_{1′}RXGNGVX₁X₂,
(SEQ ID NO: 65)
X_{4′}X_{3′}X_{2′}X_{1′}RXGNGVX₁,
(SEQ ID NO: 17)
X_{4′}X_{3′}X_{2′}X_{1′}RXGNGVX₁X₂,
(SEQ ID NO: 66)
X_{4′}X_{3′}X_{2′}X_{1′}RXGNGVX₁X₂X₃
(SEQ ID NO: 67)
X_{4′}X_{3′}X_{2′}X_{1′}RXGNGVX₁X₂X₃X₄
(SEQ ID NO: 68)
X_{4′}X_{3′}X_{2′}X_{1′}RXGNGVX₁X₂X₃X₄X₅
(SEQ ID NO: 18)
X_{4′}X_{3′}X_{2′}X_{1′}RXGNGVX₁X₂X₃X₄X₅X₆,
(SEQ ID NO: 19)
X_{5′}X_{4′}X_{3′}X_{2′}X_{1′}RXGNGVX₁X₂X₃X₄X₅X₆,
(SEQ ID NO: 20)
X_{9′}X_{8′}X_{7′}X_{6′}X_{5′}X_{4′}X_{3′}X_{2′}X_{1′}RXGNGVX₁X₂X₃X₄X₅X₆,
(SEQ ID NO: 21)
X_{8′}X_{7′}X_{6′}X_{5′}X_{4′}X_{3′}X_{2′}X_{1′}RXGNGVX₁X₂X₃X₄X₅,
(SEQ ID NO: 22)
X_{7′}X_{6′}X_{5′}X_{4′}X_{3′}X_{2′}X_{1′}RXGNGVX₁X₂X₃X₄X₅,
or
(SEQ ID NO: 23)
X_{7′}X_{6′}X_{5′}X_{4′}X_{3′}X_{2′}X_{1′}RXGNGVX₁X₂X₃X₄X₅X₆X₇,
or the inverse of any such sequences, wherein X₁, X₃, X₅, X₇, X_{8′} and X_{9′} are non-polar amino acids, and X₂, X₄, X₆, X_{1′}, X_{2′}, X_{3′}, X_{4′}, X_{5′}, X_{6′}, and X_{7′} are polar amino acids.

11. The peptide or peptidomimetic of claim 10, wherein X₁, X₂, X₃, X₄, X₅, X₆, X₇, X_{1′}, X_{2′}, X_{3′}, X_{8′} and X_{9′} are neutral amino acids, and X_{4′}, X_{5′}, X_{6′}, and X_{7′} are basic amino acids.

12. The peptide or peptidomimetic of claim 1, wherein the peptide or peptidomimetic comprises at least one of SEQ ID NOs: 24-46 or 57-61, or the inverse of any such sequences.

13. A peptide or peptidomimetic comprising at least eight contiguous amino acids of the juxtamembrane domain of IGF1R (SEQ ID NO: 43), or inverse thereof, wherein the peptide or peptidomimetic comprises a total of about 50 or fewer amino acids.

14. The peptide or peptidomimetic of claim 13, wherein the peptide or peptidomimetic consists of the juxtamembrane domain of IGF1R (SEQ ID NO: 43) or a fragment thereof.

15. The peptide or peptidomimetic of claim 13 comprising SEQ ID NO: 47 or 48, or the inverse thereof.

16. The peptide or peptidomimetic of claim 1, wherein the peptide or peptidomimetic comprises one or more D-amino acids.

17. The peptide or peptidomimetic of claim 1, wherein the peptide or peptidomimetic inhibits IGF1R dimerization in a cell.

18. The peptide or peptidomimetic of claim 1, wherein the peptide or peptidomimetic does not inhibit Insulin Receptor (IR) function.

19. The peptide or peptidomimetic of claim 1, further comprising a cell-penetrating motif.

20. The peptide or peptidomimetic of claim 1, further comprising a protein transduction domain or fatty acid, optionally attached to the peptide or peptidomimetic via a linker sequence.

21. The peptide or peptidomimetic of claim 1, wherein the peptide or peptidomimetic comprises a terminal palmitoyl group.

22. The peptide or peptidomimetic of claim 21, wherein the peptide or peptidomimetic comprises an N-terminal palmitoyl residue.

23. A nucleic acid encoding the peptide or peptidomimetic of claim 1, optionally in the form of a vector.

24. An antibody that specifically binds to the peptide or peptidomimetic of claim 1.

25. A pharmaceutical composition comprising the peptide or peptidomimetic of claim 1, a nucleic acid encoding the peptide or peptidomimetic, or an antibody that specifically binds to the peptide or peptidomimetic and a carrier.

26. A method of inhibiting IGF1R activity in a cell comprising introducing a peptide or peptidomimetic of claim 1 into the cell, whereby the activity of IGF is inhibited.

27. The method of claim 26, wherein inhibiting IGF1R activity comprises inhibiting IGF1R dimerization.

28. The method of claim 26, wherein IGF1R activity is inhibited without significantly inhibiting IR activity.

29. The method of claim 26, wherein the peptide or peptidomimetic is introduced into the cell by contacting the cell with the peptide or peptidomimetic.

30. The method of claim 26, wherein the peptide or peptidomimetic is introduced into the cell by contacting the cell with a nucleic acid encoding the peptide or peptidomimetic, whereby the peptide or peptidomimetic is expressed in the cell.

31. A method for inhibiting the growth or proliferation of a cancer cell comprising administering a peptide or peptidomimetic of claim 1 to the cancer cell, whereupon the growth or proliferation of the cancer cell is inhibited.

32. The method of claim 18, wherein the cell is in a host.

33. The method of claim 20, wherein the host is a mammal.

34. A method of treating or preventing a disease mediated by IGF1R in a host comprising administering to the host a peptide or peptidomimetic of claim 1 or nucleic acid encoding same, whereby the disease is treated or prevented.

35. The method of claim 34, wherein the disease is cancer.

36. The method of claim 34, wherein benign prostatic hyperplasia (BPH), VIPoma or Werner-Morrison syndrome, acromegaly, spinocerebellar ataxia, gigantism, psoriasis, atherosclerosis, smooth muscle restenosis of blood vessels, or abnormal microvascular proliferation.


SEQUENCE LISTING (68 sequences)

SEQ ID NO: 1 - protein, 6 aa, Artificial Sequence
  Synthetic
RXGNGV

SEQ ID NO: 2 - protein, 8 aa, Artificial Sequence
  Synthetic
RXGNGVXX

SEQ ID NO: 3 - protein, 11 aa, Artificial Sequence
  Synthetic
RXGNGVXXXXX

SEQ ID NO: 4 - protein, 12 aa, Artificial Sequence
  Synthetic
RXGNGVXXXXXX

SEQ ID NO: 5 - protein, 13 aa, Artificial Sequence
  Synthetic
RXGNGVXXXXXXX

SEQ ID NO: 6 - protein, 7 aa, Artificial Sequence
  Synthetic
XRXGNGV

SEQ ID NO: 7 - protein, 8 aa, Artificial Sequence
  Synthetic
XXRXGNGV

SEQ ID NO: 8 - protein, 9 aa, Artificial Sequence
  Synthetic
XXXRXGNGV

SEQ ID NO: 9 - protein, 10 aa, Artificial Sequence
  Synthetic
XXXXRXGNGV

SEQ ID NO: 10 - protein, 11 aa, Artificial Sequence
  Synthetic
XXXXXRXGNGV

SEQ ID NO: 11 - protein, 13 aa, Artificial Sequence
  Synthetic
XXXXXXXRXGNGV

SEQ ID NO: 12 - protein, 14 aa, Artificial Sequence
  Synthetic
XXXXXXXXRXGNGV

SEQ ID NO: 13 - protein, 15 aa, Artificial Sequence
  Synthetic
XXXXXXXXXRXGNGV

SEQ ID NO: 14 - protein, 9 aa, Artificial Sequence
  Synthetic
XRXGNGVXX

SEQ ID NO: 15 - protein, 10 aa, Artificial Sequence
  Synthetic
XXRXGNGVXX

SEQ ID NO: 16 - protein, 11 aa, Artificial Sequence
  Synthetic
XXXRXGNGVXX

SEQ ID NO: 17 - protein, 12 aa, Artificial Sequence
  Synthetic
XXXXRXGNGVXX

SEQ ID NO: 18 - protein, 16 aa, Artificial Sequence
  Synthetic
XXXXRXGNGVXXXXXX

SEQ ID NO: 19 - protein, 17 aa, Artificial Sequence
  Synthetic
XXXXXRXGNGVXXXXXX

SEQ ID NO: 20 - protein, 21 aa, Artificial Sequence
  Synthetic
XXXXXXXXXRXGNGVXXXXXX

SEQ ID NO: 21 - protein, 19 aa, Artificial Sequence
  Synthetic
XXXXXXXXRXGNGVXXXXX

SEQ ID NO: 22 - protein, 18 aa, Artificial Sequence
  Synthetic
XXXXXXXRXGNGVXXXXX

SEQ ID NO: 23 - protein, 20 aa, Artificial Sequence
  Synthetic
XXXXXXXRXGNGVXXXXXXX

SEQ ID NO: 24 - protein, 8 aa, Artificial Sequence
  Synthetic
RLGNGVLY

SEQ ID NO: 25 - protein, 9 aa, Artificial
  Synthetic
SRLGNGVLY

SEQ ID NO: 26 - protein, 10 aa, Artificial Sequence
  Synthetic
NSRLGNGVLY

SEQ ID NO: 27 - protein, 11 aa, Artificial Sequence
  Synthetic
NNSRLGNGVLY

SEQ ID NO: 28 - protein, 12 aa, Artificial Sequence
  Synthetic
RNNSRLGNGVLY

SEQ ID NO: 29 - protein, 13 aa, Artificial Sequence
  Synthetic
KRNNSRLGNGVLY

SEQ ID NO: 30 - protein, 16 aa, Artificial Sequence
  Synthetic
RNNSRLGNGVLYASVN

SEQ ID NO: 31 - protein, 17 aa, Artificial Sequence
  Synthetic
KRNNSRLGNGVLYASVN

SEQ ID NO: 32 - protein, 18 aa, Artificial Sequence
  Synthetic
RKRNNSRLGNGVLYASVN

SEQ ID NO: 33 - protein, 21 aa, Artificial Sequence
  Synthetic
VFHRKRNNSRLGNGVLYASVN

SEQ ID NO: 34 - protein, 13 aa, Artificial Sequence
  Synthetic
HRKRNNSRLGNGV

SEQ ID NO: 35 - protein, 14 aa, Artificial Sequence
  Synthetic
HRKRNNSRLGNGVL

SEQ ID NO: 36 - protein, 15 aa, Artificial Sequence
  Synthetic
HRKRNNSRLGNGVLY

SEQ ID NO: 37 - protein, 16 aa, Artificial Sequence
  Synthetic
HRKRNNSRLGNGVLYA

SEQ ID NO: 38 - protein, 17 aa, Artificial Sequence
  Synthetic
HRKRNNSRLGNGVLYAS

SEQ ID NO: 39 - protein, 19 aa, Artificial Sequence
  Synthetic
VHRKRNNSRLGNGVLYASV

SEQ ID NO: 40 - protein, 18 aa, Artificial Sequence
  Synthetic
HRKRNNSRLGNGVLYASV

SEQ ID NO: 41 - protein, 20 aa, Artificial Sequence
  Synthetic
HRKRNNSRLGNGVLYASVNP

SEQ ID NO: 42 - protein, 19 aa, Artificial Sequence
  Synthetic
HRKRNNSRLGNGVLYASVN

SEQ ID NO: 43 - protein, 26 aa, Artificial Sequence
  Synthetic
HRKRNNSRLGNGVLYASVNPEYFSAA

SEQ ID NO: 44 - protein, 13 aa, Artificial Sequence
  Synthetic
YLVGNGLRSNNRK

SEQ ID NO: 45 - protein, 13 aa, Artificial Sequence
  Synthetic
YLVGNGLRSNNRR

SEQ ID NO: 46 - protein, 15 aa, Artificial Sequence
  Synthetic
VSAYLVGNGLRSNNR

SEQ ID NO: 47 - protein, 10 aa, Artificial Sequence
  Synthetic
HRKRNNSRLG

SEQ ID NO: 48 - protein, 12 aa, Artificial Sequence
  Synthetic
HRKRNNSRLGNG

SEQ ID NO: 49 - protein, 50 aa, Artificial Sequence
  Synthetic
IALPVAVLLIVGGLVIMLYVFHRKRNNSRLGNGVLYASVNPEYFSAADVY

SEQ ID NO: 50 - protein, 50 aa, Artificial Sequence
  Synthetic
IALPVAILLIVGGLVIMLYVFHRKRNNSRLGNGVLYASVNPEYFSAADVY

SEQ ID NO: 51 - protein, 50 aa, Artificial Sequence; identical to SEQ ID NO: 50
  Synthetic

SEQ ID NO: 52 - protein, 50 aa, Artificial Sequence; identical to SEQ ID NO: 50
  Synthetic

SEQ ID NO: 53 - protein, 50 aa, Artificial Sequence
  Synthetic
VAIPLALSFLLVGIISIVCFVFKKRNSNRLGNGVLYASVNPEYFSAAEMY

SEQ ID NO: 54 - protein, 50 aa, Artificial Sequence
  Synthetic
IIIPVIVLLLLLFVIVAVIIVTKKRNSDRIGNGVLYASVNPEYFSPFEMY

SEQ ID NO: 55 - protein, 11 aa, Artificial Sequence
  Synthetic
RQPDGPLGPLY

SEQ ID NO: 56 - protein, 48 aa, Artificial Sequence
  Synthetic
IIGPLIFVFLFSVVIGSIYLFLRKRQPDGPLGPLYASSNPEYLSASDV

SEQ ID NO: 57 - protein, 12 aa, Artificial Sequence
  Synthetic
RNNSRLGNGVLF

SEQ ID NO: 58 - protein, 11 aa, Artificial Sequence
  synthetic
RNNSRLGNGVL

SEQ ID NO: 59 - protein, 13 aa, Artificial Sequence
  Synthetic
RNNSRLGNGVLYA

SEQ ID NO: 60 - protein, 14 aa, Artificial Sequence
  Synthetic
RNNSRLGNGVLYAS

SEQ ID NO: 61 - protein, 15 aa, Artificial Sequence
  Synthetic
RNNSRLGNGVLYASV

SEQ ID NO: 62 - protein, 7 aa, Artificial Sequence
  Synthetic
RXGNGVX

SEQ ID NO: 63 - protein, 9 aa, Artificial Sequence
  Synthetic
RXGNGVXXX

SEQ ID NO: 64 - protein, 10 aa, Artificial Sequence
  Synthetic
RXGNGVXXXX

SEQ ID NO: 65 - protein, 11 aa, Artificial Sequence
  Synthetic
XXXXRXGNGVX

SEQ ID NO: 66 - protein, 13 aa, Artificial Sequence
  Synthetic
XXXXRXGNGVXXX

SEQ ID NO: 67 - protein, 14 aa, Artificial Sequence
  Synthetic
XXXXRXGNGVXXXX

SEQ ID NO: 68 - protein, 15 aa, Artificial Sequence
  Synthetic
XXXXRXGNGVXXXXX